(19) United States
(12) Patent Application Publication
(10) Pub. No.: US 2018/0207443 A1
(43) Pub. Date: Jul. 26, 2018

(54) Light Irradiation Device for Teeth Whitening

(71) Applicant: Hidetoshi Nishio, Chiba (JP)

(72) Inventor: Hidetoshi Nishio, Chiba (JP)

(21) Appl. No.: 15/877,022

(22) Filed: Jan. 22, 2018

(30) Foreign Application Priority Data
Jan. 23, 2017 (JP) U2017-000204

(51) Int. Cl. A61N 5/06

(52) U.S. Cl. CPC A61N 5/0613; A61C 19/06; A61N 2005/0606

(57) ABSTRACT

The irradiation portion of the light irradiation device for teeth whitening irradiates mixed light in which far infrared rays and visible light are mixed. Visible light has a wavelength of 430 nm or more and 490 nm or less. Inside the irradiation portion, an LED which emits far infrared rays and/or an LED which emits visible light are provided.

### CROSS-REFERENCE TO RELATED APPLICATION

The present disclosure relates to subjects included in Japanese Utility Model Application 2017-000204 filed dated on Jan. 23, 2017, as a whole, the disclosure therein should be incorporated in this application explicitly by reference.

### BACKGROUND OF THE INVENTION

### 1. Field of the Invention

The present invention relates to a light irradiation device for teeth whitening used for teeth whitening in a dental clinic or a whitening salon.

### 2. Description of Related Art

In a dental clinic, not only treatment of abnormal teeth, but also whitening treatment is carried out in order to clean the normal teeth. Furthermore, in addition to doing a whitening treatment on a patient by a dentist at the dental clinic, in recent years, so-called self-whitening, which performs one's own teeth whitening treatment at a whitening salon, is widespread.

In the treatment of teeth whitening, a whitening agent such as hydrogen peroxide is introduced into the oral cavity, and the whitening agent in the oral cavity is activated by irradiating the oral cavity with visible light from a light irradiation device for whitening. The activated whitening agent decomposes dirt (stain) adhered to the teeth. (For example, see Japanese Patent Application Laid-open No. 2016-221190)

### SUMMARY OF THE INVENTION

Although visible light can activate the whitening agent to a certain extent, it is required to activate the whitening agent more effectively and to efficiently decompose the stain adhered to the teeth with the whitening agent. On the other hand, it is also conventionally required to safely perform the whitening treatment.

The present invention has been made in view of the above problems, and it is an object of the present invention to effectively activate a whitening agent, and to efficiently decompose a stain adhering to the teeth with the activated whitening agent and safely perform whitening treatment.

In order to solve the above problem, a light irradiation device (1) for teeth whitening according to the present invention includes an irradiation portion (2) for irradiating far infrared rays, an arm portion (3) one end of which is connected to the irradiation portion (2), and an attaching portion (4) provided at the other end of the arm portion (3).

According to the light irradiation device for teeth whitening of the present invention, the far infrared rays irradiated into the oral cavity of a client (a person to be treated) are absorbed by moisture in the oral cavity of the client. The far infrared light absorbed in moisture resonates with moisture, whereby the internal energy of moisture rises. As the internal energy of moisture rises, the internal temperature in the oral cavity rises. As a result, the temperature of the whitening agent charged into the oral cavity also rises. As a result, the whitening agent charged into the oral cavity of the client is activated. Stains adhered to the client's teeth are efficiently decomposed by the activated whitening agent.

Furthermore, according to the light irradiation device for whitening of the present invention, the whitening agent can be warmed up to the temperature of a human body by far infrared rays. Therefore, excessive heating of the whitening agent can be prevented. Therefore, it is possible to safely perform the whitening treatment.

Further, in the light irradiation device (1) for whitening according to the present invention, the irradiation portion (2) can irradiate mixed light in which far infrared rays and visible light are mixed. Further, the visible light may have a wavelength of 430 nm or more and 490 nm or less. In addition, an LED emitting the far-infrared ray and/or an LED emitting the visible light may be provided inside the irradiation portion (2).

It should be noted that the bracketed reference numerals are examples of the elements of the embodiments described later. Effect of the Invention

The light irradiation device for teeth whitening according to the present invention, it is possible to effectively activate the whitening agent, to efficiently decompose the stain adhering to the teeth with the activated whitening agent and safely perform the whitening.

### BRIEF DESCRIPTION OF THE DRAWINGS

FIG. 1 is an overall view of a light irradiation device according to an embodiment of the present invention;

FIG. 2 is a partially enlarged perspective view showing an irradiation portion of the light irradiation device shown in FIG. 1;

FIG. 3 is a partially enlarged perspective view showing an attaching portion of the light irradiation device shown in FIG. 1.

### DESCRIPTION OF THE EMBODIMENTS

Embodiments of the present invention will be described as below with reference to the accompanying drawings.

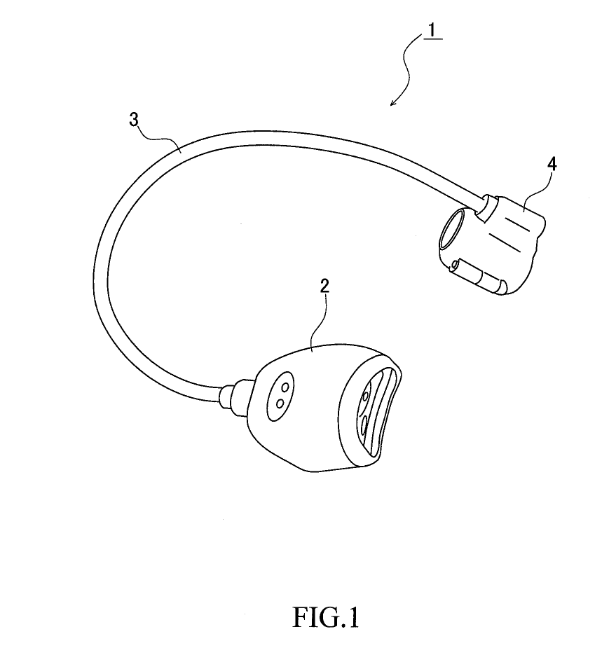

As shown in FIG. 1, a light irradiation device 1 for teeth whitening includes an irradiation portion 2 for irradiating light to a client, an arm portion 3 configured to freely bend, and an attaching portion 4 for attaching the light irradiation device 1 to the outside part of the light irradiation device 1.

The irradiation portion 2 includes a base portion 2a, an irradiation port 2b for irradiating far-infrared rays and visible light, and an operating unit 2c for switching on and off irradiation from the irradiation port 2b. The far infrared ray means an electromagnetic wave having a wavelength of 3.0 μm or more and 1000 μm or less. The visible light means an electromagnetic wave having a wavelength of 380 nm or more and 750 nm or less. Further, the light output from the irradiation port 2b can be, for example, 2500 mW/cm 2. A light emitting source (not shown) is accommodated in the irradiation portion 2. For example, an LED (Light Emitting Diode) that emits blue light having a wavelength of 430 nm or more and 490 nm or less and an LED that emits far infrared rays are accommodated in the light emitting source. The electric power of the LED emitting blue light is preferably 4 W or more and 5 W or less.

One end of the arm portion 3 is connected to the irradiation portion 2 and the other end is connected to the attaching portion 4 to connect the irradiation portion 2 and the attaching portion 4. Further, the length of the arm portion 3 can be appropriately determined according to the size of the light irradiation device 1 and the size of the place where the light irradiation device 1 is provided.

The attaching portion 4 includes a cylindrical base portion 4a, a through hole 4b passing through the base portion 4a, and a connecting portion 4c for connecting one end of a power cord (not shown). By inserting a rod-shaped portion such as a stand into the through hole 4b of the attaching portion 4, the light irradiation device 1 can be attached to this stand or the like.

It is preferable that the light irradiation device 1 has a weight of about 4.7 kg. Further, it is preferable that the light irradiation device 1 is capable of handling a power supply of 100 V or more and 240 V or less, 50 Hz or more and 60 Hz or less, 1.2 A or less.

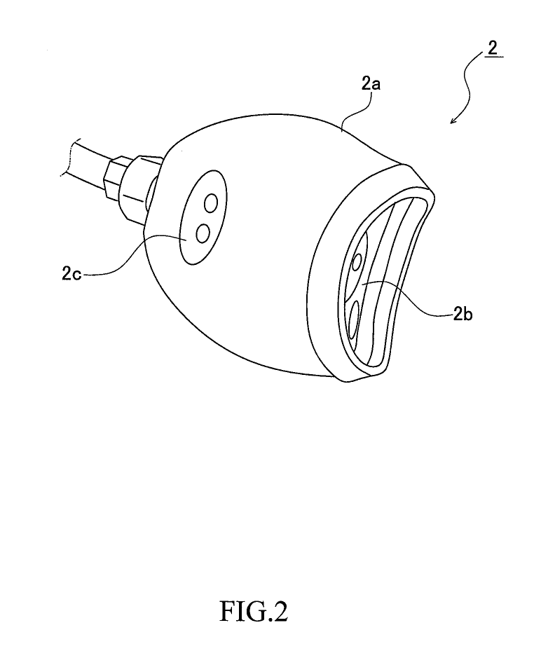

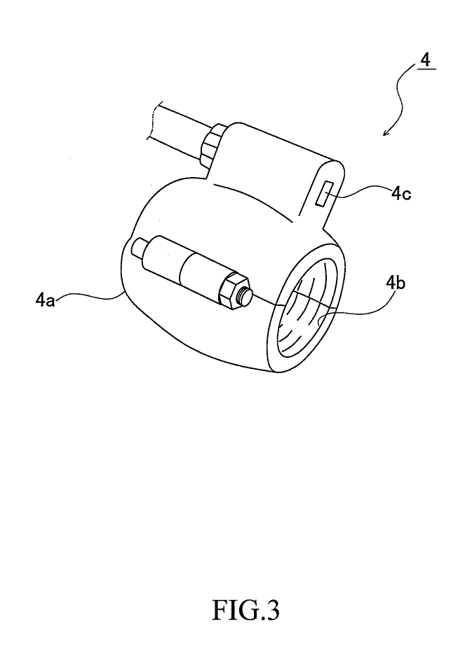

Next, the operation of the light irradiation device 1 will be described in detail with reference to FIGS. 1 to 3.

First, an operator of the lighting irradiation device 1 such as a dentist in a dental clinic or a practitioner of a whitening salon, inserts a rod-like portion such as a stand in a facility in a dental clinic into the through hole 4b of the attaching portion 4, and attach the light irradiation device 1 to a stand or the like. Further, one end of the power cord is inserted into the connecting portion 4c of the attaching portion 4 of the light irradiation device 1, and a plug provided at the other end of the power cord is plugged into an outlet.

Further, the operator applies a whitening agent to the teeth of the client such as a patient, and inserts the irradiation portion 2 of the light irradiation devices 1 into the oral cavity of the client.

Also, the operator operates the operating unit 2c of the irradiation portion 2 of the light irradiation device 1, and mixes the mixed light of the blue light and far infrared ray mixed from the irradiation port 2b of the irradiation portion 2 with the mouth of the client. The mixed light is purple.

By irradiating the mixed light into the oral cavity of the client, the far infrared rays irradiated into the oral cavity of the client are absorbed by moisture in the mouth of the client. The far infrared light absorbed in moisture resonates with moisture, whereby the internal energy of moisture rises. As the internal energy of moisture rises, the internal temperature in the oral cavity of the client rises. As a result, the temperature of the whitening agent charged into the oral cavity of the client also rises. As a result, the whitening agent introduced into the oral cavity of the client is activated, and the stain adhered to the client's teeth is efficiently decomposed by the activated whitening agent.

In addition, since the whitening agent is warmed up to the temperature of the human body by far infrared rays, it is possible to prevent the whitening agent from being excessively heated, and whitening treatment can be performed safely.

After completion of whitening treatment, the operator operates the operating unit 2c provided in the irradiation portion 2 of the light irradiation device 1, and stops irradiating light from the irradiation port 2b of the irradiation portion 2. Further, the irradiation portion 2 is pulled out from the mouth of the client, the plug of the power cord is pulled out from the receptacle, and the power cord is removed from the connecting portion 4c of the attaching portion 4. Further, the attaching portion 4 is detached from the rod-like portion such as a stand to detach the light irradiation device 1 from the stand or the like.

It is preferable to use a whitening agent containing polyphosphoric acid as a whitening agent for activating by irradiating far infrared rays with the light irradiation device 1. According to this, it is possible not only to decompose the stain adhering to the teeth of the client, but also to prevent deposition of stain on teeth. In addition, polyphosphoric acid is a substance in vivo originally present in the human body. Therefore, polyphosphoric acid can be safely used as the whitening agent. In addition, polyphosphoric acid can be warmed up to the body temperature by far infrared rays, so that the stain decomposing function can be demonstrated to the utmost. However, agents other than polyphosphoric acid can also be used as a whitening agent. For example, hydrogen peroxide, nitrogen peroxide, titanium oxide, metaphosphoric acid and the like can be used.

In the foregoing, the embodiments of the present invention have been described. However, the present invention is not limited to the above embodiments, but various modifications of the present invention are possible within the scope of the technical idea described in the claims, specification, and drawings.

What is claimed is:

1. A light irradiation device for teeth whitening comprising:
an irradiation portion which irradiates far infrared rays;
an arm portion having one end connected to the irradiation portion; and
an attaching portion connected to the other end of the arm portion.

2. The light irradiation device for teeth whitening according to claim 1, wherein;
the irradiating portion irradiates mixed light in which far infrared rays and visible light are mixed.

3. The light irradiation device for teeth whitening according to claim 2, wherein; the visible light has a wavelength of 430 nm or more and 490 nm or less.

4. The light irradiation device for teeth whitening according to claim 2, wherein; an LED emitting the far infrared ray and/or an LED emitting the visible light is provided inside the irradiation portion.

5. The light irradiation device for teeth whitening according to claim 3, wherein; an LED emitting the far infrared ray and/or an LED emitting the visible light is provided inside the irradiation portion.
